(19) United States
(12) Patent Application Publication
(10) Pub. No.: US 2009/0274742 A1
(43) Pub. Date: Nov. 5, 2009

(54) MULTIMODAL HIGH STRENGTH DEVICES AND COMPOSITES

(76) Inventor: Malcolm NMI Brown, Leeds (GB)

Correspondence Address: DIANA HOUSTON;SMITH & NEPHEW, INC., 1450 BROOKS ROAD, MEMPHIS, TN, 38116, US

(21) Appl. No.: 12/064,192

(22) PCT Filed: Aug. 16, 2006
(86) PCT No.: PCT/GB2006/003047

§ 371 Date: Jul. 8, 2008

(30) Foreign Application Priority Data
Aug. 18, 2005 (GB) 0516942.0
Nov. 16, 2005 (GB) 0523317.6

(51) Int. Cl. A61L 27/54; C08L 67/04; A61L 27/26; A61K 33/42; A61B 17/56; A61B 17/08

(52) U.S. Cl. 424/426; 525/437; 523/113; 424/602; 606/60; 606/151

(57) ABSTRACT

An oriented implantable biodegradable multimodal device is disclosed. The orientated implantable biodegradable multimodal device includes a blend of a first polymer component having a first molecular weight (mwt) together with at least a second polymer component having a mwt which is less than that of the first component. The polymer within the blend may be in a uniaxial, biaxial or triaxial orientation. Also disclosed is a composite thereof with matrix polymer, processes for the preparation thereof and the use thereof as an implantable biodegradable device such as a high strength trauma fixation device suitable for implantation into the human or animal body. As examples the high strength trauma device may take the form of plates, screws, pins, rods, anchors or scaffolds.

This application claims the benefit of U.K. Provisional Application No. 0516942.0, filed Aug. 18, 2005 and U.K. Provisional Application No. 0523317.6, filed Nov. 16, 2005 both entitled “High strength fibres and composites” and the entire contents of which are hereby incorporated by reference.

### TECHNICAL FIELD OF THE INVENTION

This invention relates to biodegradable polymeric materials, particularly to bioresorbable materials and to artifacts made therefrom.

### BACKGROUND OF THE INVENTION

High strength trauma fixation devices (plates, screws, pins etc) are presently made of metal, typically titanium and stainless steel however metal devices have several well known disadvantages.

Currently amorphous or semi-crystalline bioresorbable polymers such as polyglycolic acid (PGA) and polylactic acid (PLA) are typically used to produce low load bearing devices such as suture anchors, screws or tacks. One of the main criteria for using resorbable materials is that they carry out a mechanical function, degrade within a reasonable timeframe (for example, less than 3 years), and are ideally replaced by bone when used in bone sites. However, these materials are not used in high load bearing applications because they are not strong or stiff enough to resist deformation under high load.

To overcome these deficiencies a composite approach has been applied to generate stiffer and/or stronger bioresorbable materials. Poly (L) lactic acid (P(L)LA) fibre—composites are known. By incorporating drawn materials, such as fibres and rods in which the polymer is oriented in the direction of drawing, the directional strength is dramatically increased. Products from these combined approaches include fibre reinforced composites, using drawn fibre and a polymer matrix, and self-reinforced materials, using extruded billets which are die drawn into self reinforced rods.

However, drawn P(L)LA fibres are reported to have longer degradation times of up to 40 years.

There is, therefore, a need for improved bioresorbent fibre reinforced composites and self-reinforced materials, having a more rapid bioresorption but without reduction in initial strength and modulus.

It is well known, in the literature, that high molecular weight (mwt) polymers produce higher strength fibres than lower mwt polymers. However, it is known that high mwt polymers are more difficult to process than low mwt polymer and that they take longer to degrade. In order to balance the processing and mechanical properties requirement, polyethylene has been produced with mixed (bimodal or multimodal) molecular weight distributions. Whilst bimodal polyolefins are well known, little success has been achieved in the production of bimodal polyesters. This has been attributed to ‘transesterification’ reactions that occur in polyesters melts—this causes the scrambling of polymer chains and, thus, results in the formation of a broad single molecular weight polymer.

Surprisingly, we have managed to produce a high strength oriented bimodal polyester fibre by melt processing and orienting a polymer blend of high and low mwt polylactide, with improved processing and biodegradation properties compared to single mwt (monomodal) oriented polylactide fibre. We have also surprisingly found that this oriented bimodal fibre containing high and low mwt components has superior mechanical properties (modulus) when compared to oriented fibre produced from only the one molecular weight component.

### SUMMARY OF THE INVENTION

Thus in accordance with the broadest aspect of the present invention there is provided an oriented implantable biodegradable multimodal device comprising a blend of a first polymer component having a first molecular weight (mwt) together with at least a second polymer component having a mwt which is less than that of the first component, wherein polymer comprised within the blend is in uniaxial, biaxial or triaxial orientation.

The oriented multimodal device of the invention may comprise two polymer components of different mwt, whereby it is termed bimodal or may comprise further polymer components of respectively differing mwt. Polymer components may be of same or different polymer. Polymer components are suitably miscible.

Reference hereinafter to high mwt polymer component is to first polymer component and to low mwt polymer component is to second polymer component. Suitably the high mwt component is of conventional mwt as might be used in a monomodal polymer for high load bearing applications or may be of elevated mwt compared with such conventional usage. Suitably the low mwt component is of lower mwt than might be used in such conventional usage. Suitably the high mwt polymer component confers strength while the low mwt polymer component confers processability and enhanced degradation.

Reference herein to an oriented device is to a device comprising oriented polymer as known in the art, also known as aligned polymer, wherein the polymer is in uniaxial, biaxial or triaxial alignment. Polymers comprise discrete polymer chains which may be aligned or oriented to render the polymer in uniaxial, biaxial or triaxial alignment. Alignment or orientation is suitably conferred by further processing in suitable manner and as hereinafter defined. The oriented polymer of the invention is, therefore, distinct from polymer which has not been further processed to confer orientation, and in which polymer chains are typically in random alignment. Orientation may be determined by techniques as known in the art for example scanning electron microscopy (SEM), transmission electron microscopy (TEM), differential scanning calorimetry (DSC), X-ray, optical microscopy and the like.

Component molecular weights may be selected according to the nature of the particular polymer and the intended form and application of the device of the invention, and therefore the required strength and modulus. Respective mwts may be mwt of components prior to combining in the device of the invention, or mwt of components in situ in the oriented device. Reference herein to mwt is to Manganese (Mn). A polymer component may have a polydispersity of greater than 1, whereby reference herein to component mwt is to the mean or average mwt of the component.

In embodiments of the invention the high mwt component has mwt Mn in oriented form in the range in excess of 30,000 Daltons. In further embodiments of the invention the high mwt component has mwt Mn in oriented form in the range 50,000 to 500,000 Daltons. Molecular weight of the high mwt component for use in preparing an oriented device for higher strength applications, for example oriented fibres, may be in the range 100,000 to 400,000 Daltons. Molecular weight of the high mwt component for use in preparing an oriented device for lower strength applications, for example monoliths, may be in the range 30,000 to 130,000 Daltons. In embodiments of the invention the low mwt component has mwt Mn in oriented form in the range up to 30,000. In further embodiments of the invention the low mwt component has mwt Mn in oriented form in the range 2,000 to 30,000 Daltons.

In embodiments of the invention high mwt and low mwt components are characterized by a difference, Δ, in mwt of at least 5,000. In further embodiments of the invention Δ is at least 10,000. In other embodiments A is at least 20,000. In even further embodiments of the invention A is at least 30,000.

Suitably polymeric component is selected with an intrinsic viscosity (IV), in the range 1 to 10, and more particularly 2 to 5.

Molecular weight may be determined in known manner, for example by gel permeation chromatography (GPC), viscometry

(IV) or the like, conducted on a blend of polymer components for orienting or in an oriented device. In embodiments of the invention the oriented device of the invention displays a GPC trace comprising at least two distinguishable peaks, attributable to at least two polymer components, in addition to any artifacts or interferent peaks which may be present. The at least two peaks may be resolved or may be complex, for example peaks may not be separated by a baseline response or a main peak may comprise in addition a shoulder representing a second peak. Each peak therefore corresponds to a polydisperse polymer component having a mwt distribution about an average mwt.

The polymer components of the invention are miscible and may be capable of forming a substantially uniform blend. Due to the miscibility of the polymer components, the lower mwt polymer component plasticizes the main higher mwt polymer component. This aids flow and orientation, also known as alignment, of the multimodal polymer, and this results in enhanced mechanical properties, degradation and drawability with respect to a monomodal polymer containing only the high mwt component or only the low mwt component. Whilst it might have been expected that the low mwt component reduces the strength and stiffness (modulus) of the polymer in proportion to the amount thereof present in the polymer, we have in fact found that whilst there is a strength and modulus decrease due to low mwt, there is a compensating and overriding increase due to plasticization effect by the low mwt component of the high mwt component, aiding flow and alignment.

The low mwt and high mwt components may be present in any suitable proportions to give desired increase in strength and modulus and desired rate of degradation. In embodiments of the invention the oriented multimodal device comprises low mwt to high mwt polymer components in a mol or weight ratio of from 0.1-50:50-99.9 (low:high). In embodiments of the invention this ratio is from 0.1-30:70-99.9. The amount of each component required also depends on the polydispersity thereof and on the nature of the particular polymer or copolymer which may have differing strength, modulus and degradation properties, for example PGA, PLA, etc.

The oriented multimodal device of the invention may comprise any biodegradable polymer, including homopolymers, copolymers, blends, individual or mixed isomers and the like, which may be bioresorbable, bioerodible or display any other form of degradation, for example instability to water, heat or acid. Biodegradable polymer may be suitable for medical or other applications, for example may be suitable for implantation into the human or animal body. An oriented multimodal device of the invention may be single phase (amorphous) or biphasic (semi crystalline and amorphous). The low mwt and high mwt components may be of same or different polymer as hereinbefore defined.

Suitable biodegradable polymers are selected from:

Polyesters, including poly(lactic acid), poly(glycolic acid), copolymers of lactic and glycolic acids, copolymers of lactic and glycolic acid with poly(ethylene glycol), poly(e-caprolactone), poly(3-hydroxybutyrate), poly(p-dioxanone), poly(propylene fumarate) and mixtures thereof.

In an embodiment of the invention the oriented multimodal device of the invention comprises a polyester such as a polylactic acid, selected for example from P(L)LA, poly (D) lactic acid (P(D)LA), poly (DL) lactic acid (P(DL)LA), polycaprolactone (PLA), PGA and the like, and combinations thereof.

The polymer blend may be present as a homopolymer blend or as a co-polymer blend, including random or block copolymers or the like and including uniform or non-uniform block copolymers. In embodiments of the invention the co-polymer comprises more than one polyester or more than one isomer thereof as hereinbefore defined or comprises a polyester together with another biodegradable polymer as hereinbefore defined. For example a polymer component may comprise a polyester co-polymer of polylactic acid and glycolic acid (known as PLA/PGA co-polymer), a copolymer of P(L)LA and P(D)LA or a copolymer of P(L)LA or P(D)LA with P(DL)LA or a copolymer of PLA or an isomer thereof or of PGA with another biodegradable polymer, with a PLA or PGA copolymer or with another biodegradable copolymer as hereinbefore defined.

The polymer blend may comprise polymer components which are of the same or different polymer as hereinbefore defined. For example in the blend of the invention, in addition to any additives, both components may be a polyester or isomer thereof, such as PLA, P(L)LA, P(D)LA, or P(DL)LA, both components may be a polyester copolymer as hereinbefore defined or the like. Alternatively the blend may comprise, in addition to any additives as hereinbefore defined, high mwt P(L)LA and low mwt P(D)LA or high mwt P(D)LA and low mwt P(L)LA, high mwt P(L)LA or P(D)LA and low mwt PGA, high mwt P(L)LA or P(D)LA and low mwt copolymer as hereinbefore defined or low mwt P(L)LA or P(D)LA and high mwt copolymer as hereinbefore defined or the like.

An oriented multimodal device of the invention may incorporate additional plasticizers in the polymer blend, such as an additive which plasticizes polymer draw and which is a degradation accelerant. Such an additive may be carboxylic acid or precursor thereof such as a carboxyl containing compound, for example an acid anhydride, ester or other acid precursor. The acid may be a mono or poly saturated or unsaturated acid and is, for example, a mono or diacid. In an embodiment of the invention the acid is a monoacid or precursor thereof. The acid is suitably a C₄₋₂₄ carboxylic acid or precursor.

Lauric acid is a fatty acid known from WO 03/004071 to plasticised and accelerate the degradation of P(L)LA. We have previously found that incorporating these plasticizers increased the degree of draw of the fibres during conventional hot drawing and decreased the drawing temperature, whilst conferring more rapid bioresorption. In a particular advantage the mechanical properties were not compromised by the incorporation of plasticiser.

In embodiments of the invention the oriented multimodal device comprises a blend of first and second polymer components as hereinbefore defined in admixture, in an amount of not more than 10% by weight of the polymer components, with such an additive, for example selected from the group consisting of hexanoic acid, octanoic acid, decanoic acid, lauric acid, myristic acid, crotonic acid, 4-pentenoic acid, 2-hexenoic acid, undecylenic acid, petroselenic acid, oleic acid, erucic acid, 2,4-hexadienoic acid, linoleic acid, linolenic acid, benzoic acid, hydrocinnamic acid, 4-isopropylbenzoic acid, ibuprofen, ricinoleic acid, adipic acid, suberic acid, phthalic acid, 2-bromolauric acid, 2,4-hydroxydodecanoic acid, monobutyrin, 2-hexyldecanoic acid, 2-butyloctanoic acid, 2-ethylhexanoic acid, 2-methylvaleric acid, 3-methylvaleric acid, 4-methylvaleric acid, 2-ethylbutyric acid, trans-decanoic anhydride, lauric anhydride, myristic anhydride, 4-pentenoic anhydride, oleic anhydride, linoleic anhydride, benzoic anhydride, poly(azelaic anhydride), 2-octen-1-yl succinic anhydride and phthalic anhydride.

Aptly the polymer blend will contain not more than 5%, and more particularly not more than 2%, by weight of the additive and typically the blend will contain not more than 1% by weight of the additive. Example blends will contain not more than 2%, more particularly not more than 1%, by weight of the blend of lauric or benzoic acids or anhydrides or other precursor thereof.

The oriented multimodal device may also contain fillers such as osteoconductive materials and the like and/or biological actives such as hydroxyapatite and/or additional degradation accelerants.

The oriented multimodal device of the invention may be provided in the form of fibres, drawn monoliths, such as rods and the like, spun or moulded devices, or may be used to produce high strength composites reinforced by component fibres, drawn monoliths, spun or moulded polymer and the like. Fibres may be continuous or chopped. Reference herein to fibres includes fibres, yarns, strands, whiskers, filaments, ribbons, tapes and the like.

The oriented device of the invention is characterised by properties of high strength. In embodiments of the invention the device has a tensile strength in excess of 150 MPa up to 2000 MPa depending on the polymer components and the form thereof. Tensile strength may be in the range of about 800 to about 2000 MPa, for example 800 to 1000 or 1000 to 2000 MPa, for fibre form devices, or in the range of about 150 to about 800 MPa for drawn monoliths, spun or moulded polymer. This compares with a tensile strength of undrawn PLA fibres of the order of 70 MPa.

In a further aspect of the invention there is provided a composite comprising an oriented multimodal device as hereinbefore defined provided within a biodegradable polymer matrix.

A biodegradable polymer matrix may comprise any biodegradable polymer as hereinbefore defined and may be a homopolymer, isomer or (block) copolymer or blend thereof. In embodiments of the invention a matrix is selected from a polyester as hereinbefore defined and isomers, (block) copolymers and blends thereof as hereinbefore defined. More particularly a matrix polymer, (block) copolymer or blend is selected from one or more PLAs or isomers as hereinbefore defined.

A composite of the invention may also contain fillers such as osteoconductive materials and/or biological actives such as hydroxyapatite and/or degradation accelerants, such as acid accelerants or their precursors as hereinbefore defined, in the matrix and/or in the oriented device.

In embodiments of the invention the composite comprises oriented multimodal device present in suitable manner, for example provided as random or aligned fibres, a fabric in woven or unwoven or braided form or as a scrim, mesh, preform or prepreg. Fabrics may be mats, felts, veils, braided, knitted, punched, non-crimp, polar-, spiral- or uni-weaves, tailored fibre placement fabrics and the like. Composite may comprise continuous or chopped oriented multimodal fibres of the invention.

The oriented multimodal polymer may be present in any desired amount, for example in an amount of from about 1 wt % to about 70 wt % of the composite, and more particularly from about 5 wt % to about 30 wt %.

A composite of the invention is biodegradable and may be in the form of any implantable device where temporary residence only is required. Examples of such devices include suture anchors, soft tissue anchors, interference screws, tissue engineering scaffolds, maxillo-facial plates, fracture fixation plates and rods and the like.

The composite of the invention is characterised by properties of high strength. In embodiments of the invention the composite has a tensile strength in excess of 150 MPa up to 800 MPa depending on the constituent polymer components and matrix polymer and the composite form. Tensile strength is, for example, in the range of about 250 to about 550 MPa, for example about 350 to about 500 MPa comprising fibre form devices, drawn monoliths, spun or moulded devices.

In a further aspect of the invention there is provided a process for preparing an oriented multimodal device as hereinbefore defined comprising providing a multimodal polymer as hereinbefore defined and thereafter processing to orient multimodal polymer whereby polymer is in uniaxial, biaxial or triaxial orientation.

The preparation of multimodal polymers is known in the art. Any known method may be suitable for preparing the multimodal polymer, particularly selected from but not limited to combining one or more high mwt polymer components and one or more low mwt polymer components as hereinbefore defined for a period to allow intimate mixing thereof, or combining high mwt polymer components and catalyst for a period to allow conversion of an amount of high mwt polymer component to low mwt polymer components.

Combining polymer component and catalyst may be for a period which allows full conversion to low mwt polymer component, and thereafter combining with a further amount of high mwt polymer component, or may be for a period which is less than that which would lead to full conversion of high mwt component to low mwt component, whereby a multimodal e.g. bimodal, polymer formed in situ. For example, combining is for a period which is suitable for conversion of from about 1 wt % to about 99 wt % of high mwt component to low mwt component, from about 1 wt % to about 5 0 wt %, or from about 1 wt % to about 20 wt % of high mwt component to low mwt component.

The multimodal polymer blend used for the present invention may be produced by known processes such as solution blending wherein the components or component and catalyst are individually dissolved in a suitable solvent, for example, chloroform, and the solutions combined, by melt blending in melt phase, or by dry blending the solid polymer components or component and catalyst and subsequently or simultaneously solution blending the solid mixture with solvent such as chloroform. Any additive may be blended directly into the combined solution of the polymeric components. The solution or melt blend is then processed to form a solid multimodal blend, for example is cast onto a surface and optionally ground into particles.

In embodiments of the invention high mwt polyester component is obtained commercially or as known in the art by polymerising polyester, e.g. lactide monomer in the presence of a catalyst at elevated temperature. Low mwt polyester component is, for example, obtained commercially or as known in the art by polymerising polyester, e.g. lactide monomer in the presence of a catalyst at elevated temperature, or is obtained in situ from high mwt polymer component as hereinbefore defined.

In embodiments of the invention the blend is cast, compression moulded or extruded into a form suitable for orienting, for example monolith such as billets or rods, fibre or film, and oriented by any known process that induces orientation into a polymer as hereinbefore defined.

Casting, compression-moulding or extruding may be conducted by rendering the solid blend in melt phase for shaping into a desired form for orienting. Components may be mixed prior to rendering in melt form or may be rendered in melt form and mixed for shaping. Extrusion may be of powder or pellets as a dry blend from a single hopper or may be of component powder or pellets from individual hoppers, with in situ mixing and extrusion via a suitable die to the desired shape.

In embodiments of the invention orienting is achieved by aligning melt phase polymer and cooling, more particularly by drawing, spinning or moulding to orient polymer chains in the direction of draw or spin, or axis or direction of moulding, and cooling. For example, fibre drawing produces increased strength and modulus fibre, (hydrostatic) die drawing produces an increased strength or modulus rod or the like, spinning for example gel spinning or solution spinning produces increased strength or modulus fibre, moulding for example SCORIM (Shear Controlled Orientation in Injection Moulding) produces increased strength or modulus fibre, rod or shaped polymer, and the like. In an embodiment of the invention a high strength oriented multimodal polyester device may be produced by processing to orient the multimodal polymer using any of the following processes:—

By fibre drawing to produce a high strength-high modulus fast degrading polyester fibre (i.e. multimodal P(L)LA fibre);

By hydrostatic die drawing or die drawing to produce a high strength-high modulus fast degrading polyester rod (i.e. multimodal P(L)LA rod);

By solution processing such as gel spinning or solution spinning to produce fibre at ambient temperature from solution, with subsequent solvent removal;

By SCORIM or the like to produce monoliths with orientation by shearing effect of pistons.

Drawing, spinning and moulding processes are known in the art. In particular drawing is by feeding the fibre, film or extrudate at elevated temperature through a die and drawing the polymer whereby the polymer chains orient in the direction of drawing, and cooling. Drawing may be conducted in two stages or passes.

In one embodiment the process is for preparing an oriented multimodal device comprising polymer which is characterised by a ready chemical interactivity of polymer chains in melt phase or at elevated temperature, or which is characterised by an unstable melt phase or elevated temperature polymer blend leading to scrambling of the polymer chains and the formation of a broad single molecular weight polymer. Such polymers are typically those which are biodegradable, there being a postulated link between biodegradability and scrambling mechanisms. Previous attempts have failed to melt process multimodal polymers comprising such unstable or reactive polymers, such as polyesters, and retain multimodality.

We have now surprisingly found that it is possible to melt process a multimodal unstable or reactive polymer, such as a multimodal polyester or blends or copolymers thereof, by combining polymer components and melt processing for a period which is insufficient to allow the substantial onset of scrambling. For example, a multimodal polymer has residence time in melt phase of less than 10 minutes, for example less than 1 minute or 30 seconds, for example in the range 10 to 20 seconds, depending on the IV and the acceptable degree of scrambling thereof, e.g. has residence time in the range as defined in an extruder, for example mixing is conducted prior to melt processing, or the like. It may be appropriate to limit or monitor the melt temperature to reduce the likelihood of scrambling.

Therefore melt processing polymer components of an unstable or reactive multimodal polymer for a limited period is for a period which is sufficient to allow shaping of polymer but is insufficient to allow scrambling thereof.

Melt phase shaping is, for example, conducted at a temperature of about 200° C. to about 240° C.

The process of the invention to melt process such an unstable or reactive multimodal polymer, in particular a multimodal polyester, is of great significance. Polyesters are extremely useful polymers in the field of surgical implants due to their combined properties of high strength and modulus, together with their biodegradability. However, there is still a need to increase strength and modulus and to confer a more rapid biodegradability. The process of the invention enables for the first time the preparation of a melt processed multimodal polyester which may be useful in any polyester applications, including oriented and non-oriented applications.

In a further aspect of the invention there is therefore provided an implantable biodegradable melt processed multimodal polyester comprising a solid blend of a first polyester component having a first molecular weight together with a second polyester component having a second molecular weight which is less than the first mwt, wherein the polyester has been melt processed with retention of multimodality.

In embodiments of the invention a polyester is selected from polyester isomers, copolymers and blends thereof with polyester or other biodegradable polymers as hereinbefore defined.

In a further aspect of the invention, oriented multimodal device of the invention may be used to prepare a polymer composite as hereinbefore defined. Composites may be prepared by providing the oriented multimodal device in desired form and combining with matrix polymer as hereinbefore defined. Matrix polymer is suitably combined in solid, solution or melt form with oriented multimodal device in accordance with the invention, for example by blending, impregnation, infusion, injection or the like as known in the art, and hardened for example by moulding, compression moulding or drying.

Multimodal polymer as hereinbefore defined may be utilized to prepare both an oriented device and a matrix component of a composite material which is then fabricated into a high strength biodegradable composite device as hereinbefore defined.

In a further aspect of the invention there is provided the use of an oriented multimodal device or a composite thereof as hereinbefore defined as an implantable biodegradable device, such as a high strength trauma fixation device suitable for implantation into the human or animal body, for example plates, screws, pins, rods, anchors or scaffolds, in particular suture anchors, soft tissue anchors, interference screws, tissue engineering scaffolds, maxillo-facial plates, fracture fixation plates and rods and the like.

### BRIEF DESCRIPTION OF THE DRAWINGS

The present invention will be illustrated in non-limiting manner by reference to the following examples and accompanying figures wherein:

FIG. 1 illustrates the mwt analysis of bimodal blend of the invention; and

FIG. 2 illustrates the mwt analysis of extruded vs drawn bimodal fibre of the invention.

### DETAILED DESCRIPTION OF THE INVENTION

### Example 1

### Low Mwt PLLA Synthesis

PLLA was not readily commercially available in low mwt for use in the biomodal polyester production outlined in Example 2. Therefore a sample was prepared from monomer as outlined below.

### Methods

Preparation of Catalyst Solution in Initiator

0.0368 g of Tin (II) chloride dihydrate and 5.00 g of di(ethyleneglycol) were weighed into a 50 ml Wheaton vial. The vial was sealed then vented using a syringe needle, and the catalyst left to dissolve in the initiator.

### Preparation of Reagents

2.40 mL catalyst in initiator solution and 2.40 mL initiator were added by injection into a sealed wheaton vial containing 120 g of lactide.

### Polymerisation

The sealed vial was vented and placed in a 150° C. oven. The vial was shaken periodically as the monomer melted to mix the contents. Once the monomer had completely melted, the vial was shaken to thoroughly mix the contents and the vent removed. The vial was then transferred to a 135° C. oven. After approx. 5 days (120 hours) reaction time, the vial was removed from the oven.

### Recovery and Purification of Polymer

The hot molten polymer was poured onto clean Teflon impregnated glass cloth and allowed to cool. The cold polymer was broken into small pieces and placed in 500 ml glass jars. The polymerisation vial was rinsed with 100 mL chloroform, the washings poured into the jar with the polymer, and the jar topped up to 250 ml with fresh chloroform. A magnetic flea was added to the jar and the contents were stirred to form a clear solution. The polymer was then precipitated in approx. 10-x volume of methanol and recovered by Buchner filtration. Drying was performed in two steps, initially at 30° C. under a vacuum of 1 mbar overnight, then at 50° C. under 10-1 mbar until constant mass was obtained (around 36 hours).

### Results

92.05 g of PLLA was obtained, giving a yield of 73.6%. GPC results indicate that the average number molecular weight of this material is 3827 g/mol (polystyrene equivalent) and polydispersity of 1.32.

### Example 2

### Bimodal Polyester Production

A biomodal polyester was formed from a homogenous mixture of high molecular weight P(L)LA and the low molecular weight Poly-1-lactide formed in Example 1.

### Methods

Purasorb® Poly-1-lactide (IV=4.51) Purac lot no 0309000996
Low molecular weight Poly-1-lactide Mn=3827 g.mol⁻¹, Mw=5040 g.mol⁻¹

190 g of P(L)LA IV=4 and 10 g low molecular weight P(L)LA (from Example 1) were weighed in 500 mL glass jars. The jars were agitated by hand to homogenize the powder. The contents of Jar 1 were split into 6 jars in total, and 3.10 L of CHCl₃ were required to dissolve all the material. After 2 days agitation very viscous solutions were obtained. These were poured into 3 release paper trays. The jars were rinsed with a further 450 mL CHCl₃ and the solutions poured into a 4^{th} tray. The trays containing the polymer solution were placed in the fume cupboard for 24 h to dry. The polymer sheets were cut into rectangles (approx 10×6 cm) and dried in a vacuum oven to constant weight. After this treatment the polymers still contained 3 to 5 wt % of solvent. Thus the polymer was ground into particles (of size 3 mm). The granules were further dried under high vacuum, first at 80° C. then at 110° C. for three days until no more mass loss was observed. After drying 91% of the blend was recovered.

### Results

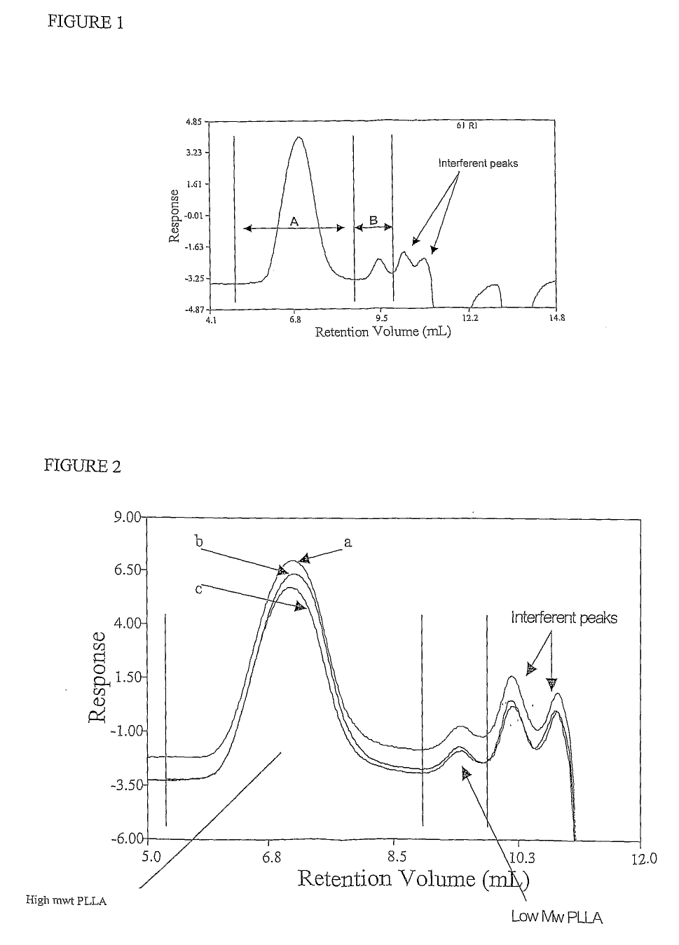

The molecular weight of the blend was determined using GPC and is shown in FIG. 1 and Table 1.

TABLE 1

| Sample | Mn | Mw | Mz | Mp | PD |
|---|---|---|---|---|---|
| Mean High MW | 361750 | 776100 | 1494000 | 542100 | 2.1 |
| fraction (A) | | | | | |
| Mean Low MW | 4700 | 5700 | 7050 | 4550 | 1.2 |
| fraction (B) | | | | | |

### Example 3

### Fibre Production

### Methods

The following methodology was used to produce both P(L)LA and bimodal P(L)LA/low mwt P(L)LA blend fibres.

The polymer (or polymer blend) was extruded using a Rondol 12 mm extruder. The extruder was fitted with:—

A general-purpose 12 mm screw (with a 25:1 L/D ratio and a 3:1 compression ratio).

A 2 mm (diameter) die (coated with lubricating coating) with a L/D ratio of 6:1.

The fibre was produced using a flat temperature profile of 240° C.

### Results

A nominal 0.5 mm diameter fibre was produced (using maximum screw speed of 50 rpm) and hauled off at a rate of 16 meters per minute. The diameter of the fibre was monitored during the run using a Mitutoyo laser micrometer. The extruded fibre was sealed in a foil pouch containing a desiccant sachet and then stored in a freezer at −20° C. prior to further processing.

### Example 4

### Drawn Fibre

### Methods

The following methodologies were used to draw both P(L)LA and bimodal P(L)LA/low mwt fibres.

### Hot Shoe

Fibre drawing was carried out using a customised drawing rig. The rig consists of two sets of godets and heated plate (hot shoe). The godets were preset to rotate at different speeds. The fibre was feed from a spindle, through the 1^{st} set of godets, drawn over the hot shoe and around the 2^{nd} set of godets. The drawn fibre was finally collected on a Leesona fibre winder. The fibres were drawn under various conditions to produce fibres with different properties.

These drawn fibres were mechanically tested, using the Instron 5566 with a crosshead speed of 10 mm/min and a gauge length of 35 mm. The fibre diameter was measured using callipers and micrometers. The total draw ratio (defined as the square of initial fibre diameter/square of final fibre diameter) is given with the strength and modulus data in Table 2 below.

TABLE 2

| | | | | | | | | |
|---|---|---|---|---|---|---|---|---|
| | Speed of | Speed of | | Speed of | Speed of | Fibre | Max | |
| Temp 1^{st} | Godet 1 | Godet 2 | Temp 2^{nd} | Godet 1 | Godet 2 | Dia | stress | Modulus |
| pass (C) | (RPM) | (RPM) | pass (C) | (RPM) | (RPM) | (mm) | (MPa) | (GPa) |

a) PLLA/+low mwt PLLA

| | | | | | | | | |
|---|---|---|---|---|---|---|---|---|
| 160 | 4 | 16 | — | — | — | 0.177 | 532 | 9.81 |
| 160 | 4 | 16 | 180 | 10 | 16 | 0.173 | 784 | 9.93 |
| 160 | 4 | 16 | 180 | 10 | 17.5 | 0.133 | 1084 | 11.53 |

b) PLLA

| | | | | | | | | |
|---|---|---|---|---|---|---|---|---|
| 160 | 4 | 16 | | | | 0.225 | 429 | 6.89 |
| 180 | 4 | 16 | — | — | — | 0.215 | 397 | 6.26 |
| 180 | 3 | 21 | — | — | — | 0.160 | 700 | 6.21 |
| 185 | 3 | 21 | — | — | — | 0.147 | 782 | 7.61 |
| 200 | 5 | 35 | — | — | — | 0.223 | 413 | 5.41 |
| 160 | 4 | 16 | 180 | 10 | 20 | 0.152 | 890 | 8.20 |
| 180 | 4 | 16 | 180 | 10 | 19 | 0.132 | 1112 | 8.72 |

### Example 5

### MWT Analysis of Extruded and Drawn Fibre

In FIG. 2 Sample a) is extruded fibre, Sample b) is extruded and drawn fibre (single drawn), Sample c) is extruded fibre and drawn fibre (double drawn). The results show that mwt of single and double drawn polymer is substantially the same as that of undrawn polymer, and that the bimodality appears to be substantially unaffected by drawing. This provides for the first time a melt processed multimodal polyester, there is evidence that scrambling has not taken place, contrary to all previous attempts in the prior art.

In view of the foregoing, it will be seen that the several advantages of the invention are achieved and attained.

The embodiments were chosen and described in order to best explain the principles of the invention and its practical application to thereby enable others skilled in the art to best utilize the invention in various embodiments and with various modifications as are suited to the particular use contemplated.

As various modifications could be made in the methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

1. An oriented implantable biodegradable multimodal device comprising a blend of a first polymer component having a first molecular weight and at least a second polymer component having a second molecular weight wherein the second molecular weight is less than the first molecular weight, and wherein the first and second polymer components comprised within the blend are in uniaxial, biaxial or triaxial orientation.

2. (canceled)

3. The oriented multimodal device of claim 1 wherein the first polymer component has a number average molecular weight (Mn) in oriented form in excess of about 30,000 Dalton and the second polymer component has a number average molecular weight (Mn) in oriented form of up to about 30,000 Dalton.

4. The oriented multimodal device of claim 3, wherein the first polymer component has a number average molecular weight (Mn) in oriented form of about 50,000 to about 500,000 Dalton and the second polymer component has a number average molecular weight (Mn) in oriented form of about 2,000 to about 30,000 Dalton.

5. The oriented multimodal device of claim 1 wherein the first and second polymeric components each have intrinsic viscosity (IV), in the range of 1 to 10.

6. The oriented multimodal device of claim 1 wherein the device displays a GPC trace comprising at least two distinguishable peaks, attributable to at least two polymer components, in addition to any artifacts or interferent peaks which may be present.

7. The oriented multimodal device of claim 1 wherein the first and second polymer components are present in a mol or weight ratio of from about 50-99.99:0.1-50 (first:second).

8. The oriented multimodal device of claim 1 wherein the first and second polymer components are present in a mol or weight ratio of from about 70-99.9:0.1-30 (first:second).

9. The oriented multimodal device of claim 1 wherein at least one of the first and second polymer components is a biodegradable, bioresorbable, or degradable homopolymer, copolymer, blend, or individual or mixed isomer selected from the group consisting of:
polyesters, poly(lactic acid), poly(glycolic acid), copolymers of lactic and glycolic acids, copolymers of lactic and glycolic acid with poly(ethylene glycol), poly(e-caprolactone), poly(3-hydroxybutyrate), poly(p-dioxanone), and poly(propylene fumarate) and combinations thereof.

10. The oriented multimodal device of claim 1 wherein at least one of the first and second polymer components comprises a polyester, selected from the group consisting of poly (L) lactic acid, poly (D) lactic acid, poly (DL) lactic acid, polycarprolactone, polyglycolic acid, and combinations thereof.

11. The oriented multimodal device of claim 1 wherein the first and second polymer components independently comprise a copolymer selected from the group consisting of polyester co-polymers of lactic acid and glycolic acid, co-polymers of poly (L) lactic acid and poly (D) lactic acid, co-polymers of poly (L) lactic acid and poly (DL) lactic acid, copolymers of poly (D) lactic acid and poly (DL) lactic acid, co-polymers of poly (L) lactic acid and a biodegradable polymer; co-polymers of poly (D) lactic acid and a biodegradable polymer, and combinations thereof.

12. The oriented multimodal device of claim 1 comprising first and second polymer components both comprising a polyester or isomer thereof or a polyester copolymer.

13. (canceled)

14. The oriented multimodal device of claim 1 comprising a solid blend of first and second polymer components in admixture, in an amount of not more than 10% by weight of the polymer components; and an additive which plasticizes polymer draw and which is a degradation accelerant.

15. The oriented multimodal device of claim 1 comprising a filler and/or a biological active and/or a degradation accelerant.

16. The oriented multimodal device of claim 1 wherein the device is a fiber or a drawn monolith.

17. A composite comprising a biodegradable polymer matrix comprising the oriented multimodal device of claim 1.

18. The composite of claim 17 wherein the polymer matrix comprises a biodegradable homopolymer, copolymer, blend, or individual or mixed isomer selected from the group consisting of:
polyesters, poly(lactic acid), poly(glycolic acid), copolymers of lactic and glycolic acids, copolymers of lactic and glycolic acid with poly(ethylene glycol), poly(e-caprolactone), poly(3-hydroxybutyrate), poly(p-dioxanone), poly(propylene fumarate) and combinations thereof.

19. The composite of claim 17 wherein the matrix further comprises a filler and/or a biological active and/or a degradation accelerant.

20. A process for preparing an oriented multimodal device comprising the steps of:
i) providing a multimodal polymer comprising a blend of a first polymer component having a first molecular weight and at least a second polymer component having a second molecular weight wherein the second molecular weight is less than the first molecular weight; and,
ii) processing the blend to orient the multimodal polymer, whereby the multimodal polymer is in uniaxial, biaxial or triaxial orientation.

21. (canceled)

22. The process of claim 20 wherein the multimodal polymer is obtained by combining one or more first polymer components and one or more second polymer components for a period of time to allow mixing thereof.

23. The process of claim 20, further comprising the steps of (i) forming the polymer by casting, compression moulding, or extruding, whereby the polymer is formed into a monolith, fibre, or film; (ii) orienting the polymer chains by aligning melt phase polymer, drawing, spinning, or moulding, wherein the polymer chains are oriented in the direction of draw, spin, or axis or direction of moulding; and (iii) cooling.

24. The process of claim 20, wherein the processing step comprises a melt processing step wherein the multimodal polymer is melt processed for a period of time which is insufficient to allow the substantial onset of scrambling, and wherein the multimodal polymer is an unstable or reactive multimodal polymer.

25. An implantable biodegradable melt processed multimodal polyester comprising a solid blend of a first polyester component having a first molecular weight and a second polyester component having a second molecular weight wherein the second molecular weight is less than the first molecular weight, and wherein the polyester has been melt processed with retention of multimodality.

26. A process for preparing a composite comprising the process of claim 20, further comprising the step of combining the oriented multimodal device with a matrix polymer.

27. A method of treating a trauma comprising implanting the device of claim 1 into a human or animal bed.

28. The method of claim 27, wherein the device is selected from the group consisting of plates, screws, pins, rods, anchors or scaffolds.

29. The method of claim 27, wherein the device is selected from the group consisting of suture anchors, soft tissue anchors, interference screws, tissue engineering scaffolds, maxillo-facial plates, fracture fixation plates or rods.

30. The oriented multimodal device of claim 9, wherein the first and second polymer components independently comprise poly (L) lactic acid, poly (D) lactic acid, or a copolymer selected from the group consisting of polyester co-polymers of lactic acid and glycolic acid, co-polymers of poly (L) lactic acid and poly (D) lactic acid, co-polymers of poly (L) lactic acid and poly (DL) lactic acid, copolymers of poly (D) lactic acid and poly (DL) lactic acid, co-polymers of poly (L) lactic acid and a biodegradable polymer; co-polymers of poly (D) lactic acid and a biodegradable polymer, and combinations thereof.

31. The process of claim 24 wherein the multimodal polymer comprises a polyester.

32. The oriented multimodal device of claim 15, wherein the filler comprises an osteoconductive material.

33. The oriented multimodal device of claim 15, wherein the biological active comprises hydroxyapatite.

34. The composite of claim 17, wherein the oriented device further comprises a filler and/or a biological active and/or a degradation accelerant.

35. The composite of claim 19, wherein the filler comprises an osteoconductive material.

36. The composite of claim 19, wherein the biological active comprises hydroxyapatite

37. The process of claim 20, wherein the first polymer component is a high molecular weight polymer component and the multimodal polymer is obtained by combining one or more first polymer components and catalyst for a period of time to allow conversion of an amount of first polymer component to a low molecular polymer component.
